(19) United States
(12) Patent Application Publication
(10) Pub. No.: US 2011/0118835 A1
(43) Pub. Date: May 19, 2011

(54) BRANCHED OCULAR IMPLANT

(76) Inventors: Matthew Silvestrini, Minneapolis, MN (US); Thomas A. Silvestrini, Alamo, CA (US)

(21) Appl. No.: 12/852,407

(22) Filed: Aug. 6, 2010

Related U.S. Application Data
(60) Provisional application No. 61/233,798, filed on Aug. 13, 2009.

(51) Int. Cl. A61F 2/16

(52) U.S. Cl. 623/6.14

(57) ABSTRACT

Disclosed is a glaucoma treatment device having a tubular, unbranched portion and a branched portion. The unbranched portion has an internal flow pathway extending between a distal region and a proximal region. The branched portion extends from the distal region of the tubular, unbranched portion. The proximal region of the unbranched portion includes at least one inflow port that is in fluid communication with the internal flow pathway. The branched portion includes a first and a second reversibly deformable branch. Each of the first and second branches has at least one outflow port and an internal lumen in fluid communication with the flow pathway of the unbranched portion. Each of the first and second branches is biased away from one another when in a relaxed state.

### REFERENCE TO PRIORITY DOCUMENT

This application claims the benefit of priority under 35 U.S.C. §119(e) of U.S. Provisional Patent Application Ser. No. 61/233,798, filed Aug. 13, 2009, under 37 C.F.R. §1.78(a). Priority of the filing date is hereby claimed and the full disclosure of the aforementioned application is incorporated herein by reference.

### BACKGROUND

This disclosure relates generally to methods and devices for use in treating glaucoma. The mechanisms that cause glaucoma are not completely known. It is known that glaucoma results in abnormally high pressure in the eye, which leads to optic nerve damage. Over time, the increased pressure can cause damage to the optic nerve, which can lead to blindness. Treatment strategies have focused on keeping the intraocular pressure down in order to preserve as much vision as possible over the remainder of the patient's life.

Past treatment includes the use of drugs that lower intraocular pressure through various mechanisms. The glaucoma drug market is an approximate two billion dollar market. The large market is mostly due to the lack of effective surgical alternatives that are long lasting and complication-free. Unfortunately, drug treatments as well as surgical treatments that are available need much improvement, as they can cause adverse side effects and often fail to adequately control intraocular pressure. Moreover, patients are often lackadaisical in following proper drug treatment regimens, resulting in a lack of compliance and further symptom progression.

With respect to surgical procedures, one way to treat glaucoma is to implant a drainage device in the eye. The drainage device functions to drain aqueous humor from the anterior chamber and thereby reduce the intraocular pressure. The drainage device is typically implanted using an invasive surgical procedure. Pursuant to one such procedure, a flap is surgically formed in the sclera. The flap is folded back to form a small cavity and the drainage device is inserted into the eye through the flap. Such a procedure can be quite traumatic as the implants are large and can result in various adverse events such as infections and scarring, leading to the need to re-operate.

Current devices and procedures for treating glaucoma have disadvantages and only moderate success rates. The procedures are very traumatic to the eye and also require highly accurate surgical skills, such as to properly place the drainage device in a proper location. In addition, the devices that drain fluid from the anterior chamber to a subconjunctival bleb beneath a scleral flap are prone to infection, and can occlude and cease working. This can require re-operation to remove the device and place another one, or can result in further surgeries. In view of the foregoing, there is a need for improved devices and methods for the treatment of glaucoma.

### SUMMARY

There is a need for improved devices and methods for the treatment of eye diseases such as glaucoma.

In one aspect, disclosed is a glaucoma treatment device having a tubular, unbranched portion and a branched portion. The unbranched portion has an internal flow pathway extending between a distal region and a proximal region. The branched portion extends from the distal region of the tubular, unbranched portion. The proximal region of the unbranched portion includes at least one inflow port that is in fluid communication with the internal flow pathway. The branched portion includes a first and a second reversibly deformable branch. Each of the first and second branches has at least one outflow port and an internal lumen in fluid communication with the flow pathway of the unbranched portion. Each of the first and second branches is biased away from one another when in a relaxed state.

In a variation the branched portion can be defined by a fractal design. The fractal design can be a self-repetitive fractal design. At least one of the first and second branches of the device can include a third and a fourth branch that are reversible deformable and have at least one outflow port and an internal lumen in fluid communication with the flow pathway of the unbranched portion. The internal lumen of each of the third and fourth branches can have a diameter that is smaller than a diameter of the branch from which it originated. The reversibly deformable branches can be constrained such that an outer dimension of the branched portion approaches an outer dimension of the unbranched portion. The inflow port can be positioned in fluid communication with an anterior chamber of the eye and the at least one outflow port can be positioned within a region outside the anterior chamber. The at least one outflow port can be positioned at a distal end of the first and second branches. The outflow port can be positioned near a distal end of the first and second branches. The first and second branches can have more than one outflow port.

Other features and advantages should be apparent from the following description of various embodiments, which illustrate, by way of example, the principles of the invention.

### BRIEF DESCRIPTION OF THE DRAWINGS

FIG. 1A is a cross-sectional, perspective view of a portion of the eye showing the anterior and posterior chambers of the eye;

FIG. 1B is a cross-sectional view of a human eye;

FIG. 2A is a side view of a branched implant collapsed inside a delivery tube;

FIG. 2B is a side view of the branched implant of FIG. 2A in the spread apart configuration;

FIG. 2C is a schematic view of a branched implant illustrating migration resistance;

FIG. 3 is a side view of a branched implant having sealed or closed ends;

FIGS. 4A-4D illustrate branched implants having expandable portions;

FIGS. 5A-5B illustrate implantation of a branched implant;

FIG. 6 illustrates an exemplary delivery system that can be used to deliver a branched implant into the eye;

FIG. 7A shows the branched implant positioned within a distal region of the delivery tube;

FIG. 7B shows the delivery system positioned for penetration into the eye;

FIG. 8 shows an enlarged view of the anterior region of the eye with a portion of the delivery system positioned in the anterior chamber;

FIG. 9 shows the distal tip of a delivery system positioned within the suprachoroidal space.

### DETAILED DESCRIPTION

Many structures in nature are fractal and branch repeatedly into smaller, self-repetitive units like branches of a tree or vessels of the cardiovascular system. Such fractal systems maximize the surface area for transport inside of a finite volume. The glaucoma implants described herein incorporate a branched portion defined by a fractal design of extended, self-similar branches over a finite scale such that they resist migration and improve fluid flow out of an implanted location in the eye, such as the anterior chamber. As will be described in more detail below, the branched implants described herein can be implanted into the eye to drain fluid from a first location to a second location, such as from the anterior chamber to the suprachoroidal space or to a filtration flap or bleb. The implants branch fractally in a bifurcation pattern. For example, each bifurcation can be self-repetitive and have a diameter smaller than the diameter of the segment from which it branched. The fractal pattern can be symmetrical or non-symmetrical. The entire branched implant can collapse into a cylindrical package such that it fits inside a delivery tube having an inner diameter just greater than the outer diameter of the largest implant portion. The branched implant collapsed within the delivery tube can be implanted in a minimally-invasive manner through a narrow channel. After insertion, removal of the delivery tube allows the collapsed branches of the implant to spread apart into a deployed state, or spread apart configuration. The open structure or spread apart configuration of the branched implant can provide a fractal flow field for fluid outflow from the anterior chamber and transport into a larger surface field, such as within the suprachoroidal space. The spread apart configuration of the branched implant can also resist migration both proximally and distally.

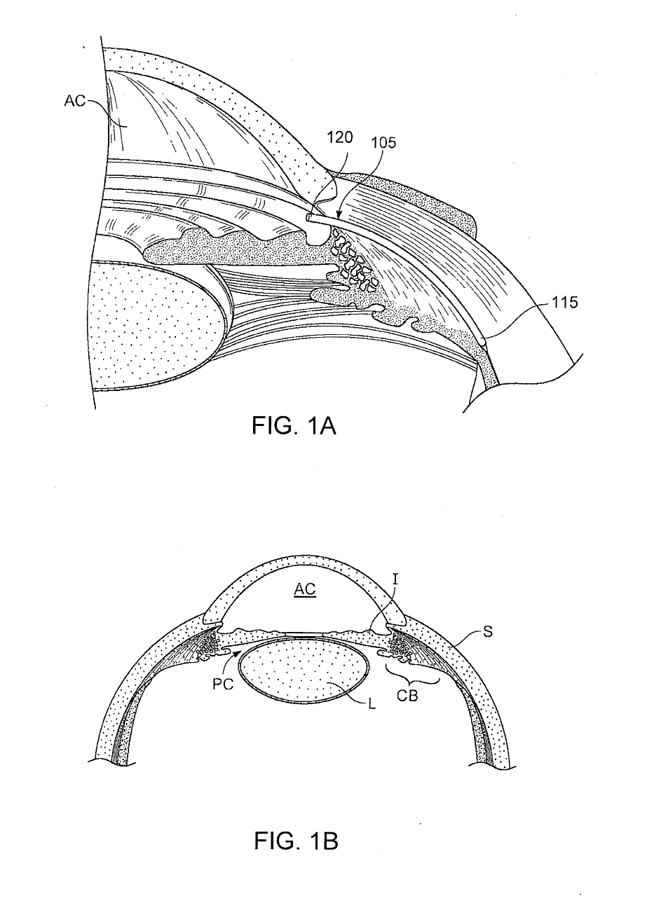

FIG. 1A is a cross-sectional, perspective view of a portion of the eye showing the anterior and posterior chambers of the eye. A schematic representation of an implant 105 is positioned inside the eye such that a proximal end 120 is located in the anterior chamber AC and a distal, branched end extends to a region of the eye that is between the ciliary body and the sclera. Alternatively, the distal end 115 can extend to a region of the eye that is posterior to the ciliary body, such as between the choroid and the sclera. The suprachoroidal space (sometimes referred to as the perichoroidal space) can include the region between the sclera and the choroid. The suprachoroidal space can also include the region between the sclera and the ciliary body. In this regard, the region of the suprachoroidal space between the sclera and the ciliary body may sometimes be referred to as the supraciliary space. The implants described herein are not necessarily positioned between the choroid and the sclera. The implants can be positioned at least partially between the ciliary body and the sclera or they can be at least partially positioned between the sclera and the choroid. In any event, the implants can provide a fluid pathway for flow of aqueous humor through or along the implant between the anterior chamber and the suprachoroidal space. In an embodiment, the proximal end is unbranched although the proximal end may be branched in other embodiments. For clarity of illustration, the distal, branched portions of the implant are not illustrated in FIG. 1A.

FIG. 1B is a cross-sectional view of a portion of the human eye. The eye is generally spherical and is covered on the outside by the sclera S. The retina lines the inside posterior half of the eye. The retina registers the light and sends signals to the brain via the optic nerve. The bulk of the eye is filled and supported by the vitreous body, a clear, jelly-like substance. The elastic lens L is located near the front of the eye. The lens L provides adjustment of focus and is suspended within a capsular bag from the ciliary body CB, which contains the muscles that change the focal length of the lens. A volume in front of the lens L is divided into two by the iris I, which controls the aperture of the lens and the amount of light striking the retina. The pupil is a hole in the center of the iris I through which light passes. The volume between the iris I and the lens L is the posterior chamber PC. The volume between the iris I and the cornea is the anterior chamber AC. Both chambers are filled with a clear liquid known as aqueous humor.

The ciliary body CB continuously forms aqueous humor in the posterior chamber PC by secretion from the blood vessels. The aqueous humor flows around the lens L and iris I into the anterior chamber and exits the eye through the trabecular meshwork, a sieve-like structure situated at the corner of the iris I and the wall of the eye (the corner is known as the iridocorneal angle). Some of the aqueous humor filters through the trabecular meshwork near the iris root into Schlemm's canal, a small channel that drains into the ocular veins. A smaller portion rejoins the venous circulation after passing through the ciliary body and eventually through the sclera (the uveoscleral route).

Glaucoma is a disease wherein the aqueous humor builds up within the eye. In a healthy eye, the ciliary processes secrete aqueous humor, which then passes through the angle between the cornea and the iris. Glaucoma appears to be the result of clogging in the trabecular meshwork. The clogging can be caused by the exfoliation of cells or other debris. When the aqueous humor does not drain properly from the clogged meshwork, it builds up and causes increased pressure in the eye, particularly on the blood vessels that lead to the optic nerve. The high pressure on the blood vessels can result in death of retinal ganglion cells and eventual blindness.

Closed angle (acute) glaucoma can occur in people who were born with a narrow angle between the iris and the cornea (the anterior chamber angle). This is more common in people who are farsighted (they see objects in the distance better than those which are close up). The iris can slip forward and suddenly close off the exit of aqueous humor, and a sudden increase in pressure within the eye follows.

Open angle (chronic) glaucoma is by far the most common type of glaucoma. In open angle glaucoma, the iris does not block the drainage angle as it does in acute glaucoma. Instead, the fluid outlet channels within the wall of the eye gradually narrow with time. The disease usually affects both eyes, and over a period of years the consistently elevated pressure slowly damages the optic nerve.

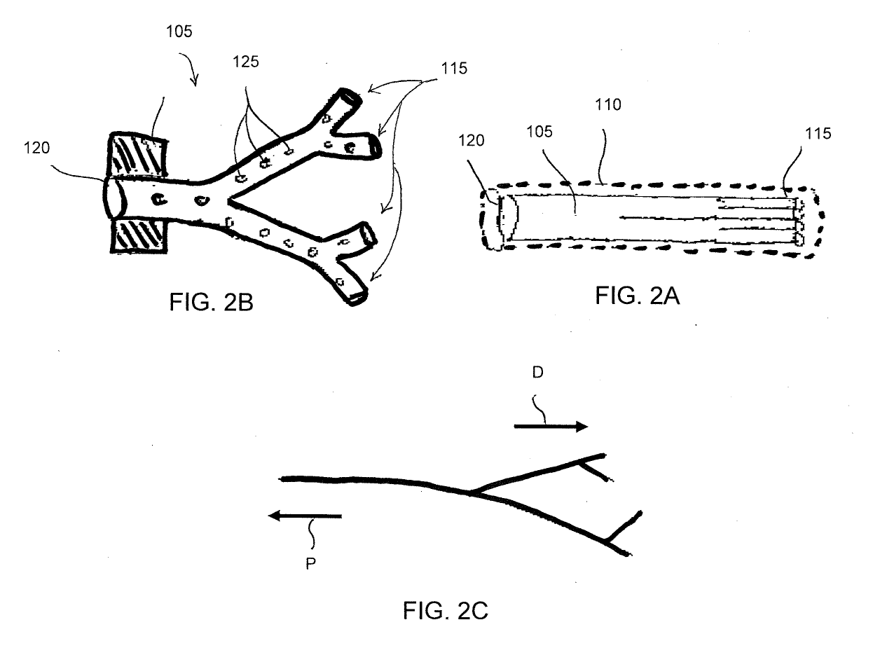

An embodiment of a branched implant 105 is shown in FIG. 2A collapsed inside a delivery tube 110. FIG. 2B shows an example of the branched implant 105 upon removal of the delivery tube 110 in a spread apart configuration. The implant 105 can include a number of self-repeating branches each with confluent internal lumens and at least one opening for ingress of fluid (such as aqueous humor from the anterior chamber) and at least one opening for egress of fluid. The number of self-repeating branches of an implant 105 can vary. In an embodiment, the branched implant 105 can have at least two self-repeating branches. In another embodiment, the branched implant 105 can have at least three total branches. It should be appreciated that any number of self-repeating branches or total branches are considered herein. The shape of the branched implant 105 can vary along its length. The implant 105 can have various cross-sectional shapes (such as a circular, oval or rectangular shape) and can vary in cross-sectional shape moving along its length. As shown in FIG. 2C, the branched implant 105 can resist migration in both the proximal direction (arrow P) and distal direction (arrow D) within a tissue channel.

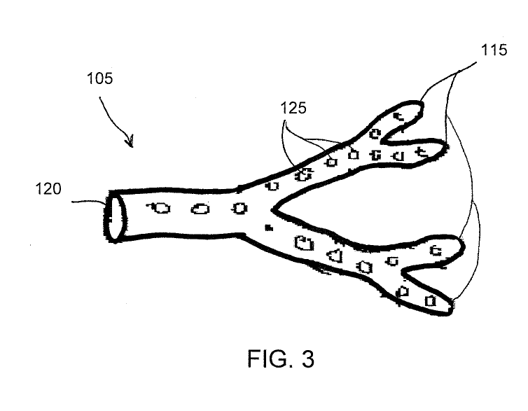

The distal ends 115 of the implant 105 can be sealed or closed to prevent plugging or jamming during insertion (see FIG. 3). In such an embodiment, drainage through the closed end implant 105 could be maintained by openings 125 drilled through the walls of the implant 105. Alternately, the openings can be initially blocked or sealed and then opened during or after implantation such as by ablating with a laser. The implant 105 can include various arrangements of openings 125 that communicate with the lumen(s). The openings 125 in the implant 105 can be filled with a material or mixture of materials, such as a sponge material, to prevent unwanted tissue in-growth into the openings 125 when the implant 105 is positioned in the eye. The sponge material can also be filled with a drug or other material that leaches into the eye over time upon implantation. During delivery of the implant 105, the openings 125 can be positioned so as to align with predetermined anatomical structures of the eye. For example, one or more openings 125 can align with the suprachoroidal space to permit the flow of aqueous humor into the suprachoroidal space, while another set of openings 125 can align within structures proximal to the suprachoroidal space, such as structures in the ciliary body or the anterior chamber of the eye.

The branches of the implant 105 can be reversibly deformed such that they can take on a narrow profile that is suitable for insertion through a small opening and then return to the spread apart configuration for retention in eye tissue and fluid passage from the anterior chamber. The implant 105 can be maintained in the insertion shape when it is under a tension or constrained in some manner such as by a delivery tube having a lower flexibility or elasticity than the branches of the implant 105. When the implant is at or near the desired location in the eye, the constraint(s) can be removed or released so that the branches of the implant revert or transition back to their spread apart configuration.

The mechanism by which the branches spread apart can vary. In one embodiment, the implant and/or the branches of the implant can be heat-set or pressure-set such that the branches have a tendency to return to a particular shape upon removal of the delivery tube. Alternately, the spring-open action can be provided by coating or manufacturing the branched portions out of a thermoplastic elastomer (TPE) that is capable of being reversibly deformed. In another embodiment, the branches can be at least partially formed of a shape-change material that changes shape in response to predetermined conditions, such as a change in temperature.

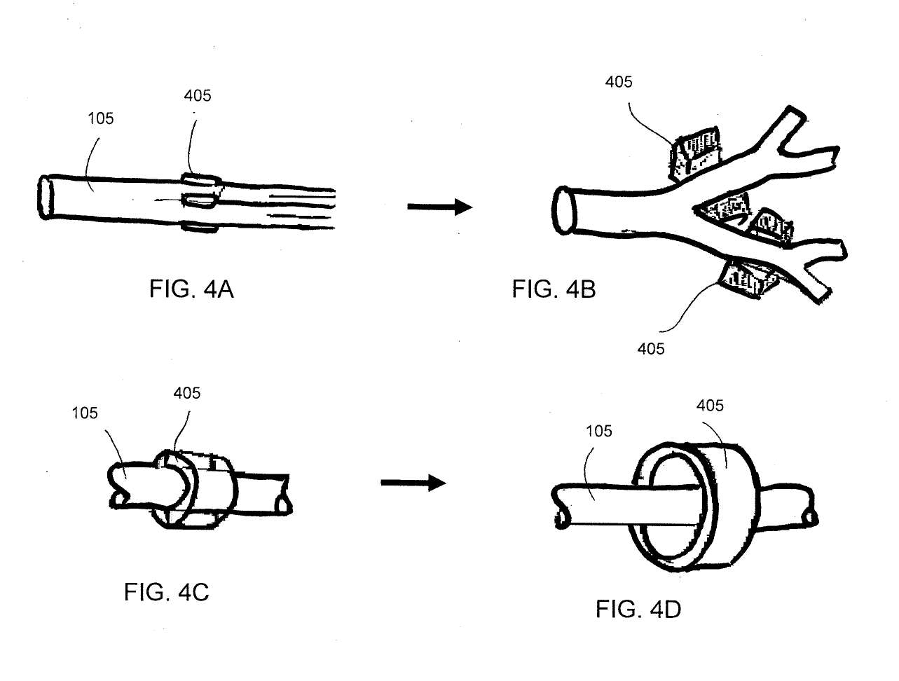

Portions of the branched implant 105 can also be coated with unhydrated Hydrogel 405 (see in FIGS. 4A-4D). Upon implantation and exposure to fluid, the Hydrogel can expand and urge the branches away from one another. In an embodiment, the Hydrogel 405 can form rings around one or more of the branches. The unhydrated Hydrogel 405 can have a low profile such that the implant 105 can be collapsed and delivered through a delivery tube. Upon removal of the delivery tube, the Hydrogel 405 can become hydrated and the rings can expand urging the branches away from one another.

The implant 105 or portion(s) thereof can be made of various materials, including, for example, thermoplastic elastomers, polyimide, Nitinol, platinum, stainless steel, molybdenum, or any other suitable polymer, metal, metal alloy, or ceramic biocompatible material or combinations thereof. The material of manufacture is desirably selected to have material properties suited for the particular function of the implant or portion thereof. In an embodiment the implant 105 is manufactured of a memory polymer. The implant 105 can also include one or more portions that are manufactured of different materials. As an example, the branched portion can be manufactured of a material different than the unbranched portion of the implant.

The implant 105 can also be manufactured of wire coils. As an example, four wire coils can be coiled together. These wire coils can separate and branch into two arms having two coils each. These arms can further branch into four arms with one coil each. The coiled structure can be coated to form a branched tubular structure reinforced with the wire coils. Different polymers can be used to coat different sections, branches or arms of the coiled structure.

Wires and polymers can be memory-formed such that the branches of the implant separate “out of plane” after they are deployed from the delivery tube to create a space, for example by pushing out the suprachoroidal space to form a lake. In an embodiment, one branch of the implant can separate in an upwards direction and another branch of the implant can separate in a downwards direction. A variety of axes and planes for separation of the branches are considered.

The implant can have braids or wires reinforced with polymer, Nitinol, or stainless steel braid or coiling or can be a co-extruded or laminated tube with one or more materials that provide acceptable flexibility and hoop strength for adequate lumen support and drainage through the lumen. The implant 105 can be enhanced by reinforcements of deformable superelastic braids or coils made from Nitinol or similar materials with enough spring-back memory (i.e. stainless spring steel).

Other materials of manufacture or materials with which the implant can be coated or manufactured entirely include any biocompatible thermoplastic polymer, any biocompatible thermo set or other wise cross-linked polymer, silicone, thermoplastic elastomers (HYTREL, KRATON, PEBAX), certain polyolefin or polyolefin blends, elastomeric alloys, polyurethanes, thermoplastic copolyester, polyether block amides, polyamides (such as Nylon), block copolymer polyurethanes (such as LYCRA). Some other exemplary materials include fluoropolymers (such as FEP and PVDF), polyester, ePTFE (also known as GORETEX), FEP laminated into nodes of ePTFE, acrylic, low glass transition temperature acrylics, silver coatings (such as via a CVD process), gold, polypropylene, poly(methyl methacrylate) (PMMA), PolyEthylene Terephthalate (PET), Polyethylene (PE), PLLA, parylene, PEEK, polysulfone, polyamideimides (PAI) and liquid crystal polymers. It should also be appreciated that stiffer polymers can be made to be more compliant by incorporating air or void volumes into their bulk, for example, PTFE and expanded PTFE. In order to maintain a low profile, well-known sputtering techniques can be employed to coat the implant. Such a low profile coating would accomplish a possible goal of preventing migration while still allowing easy removal if desired.

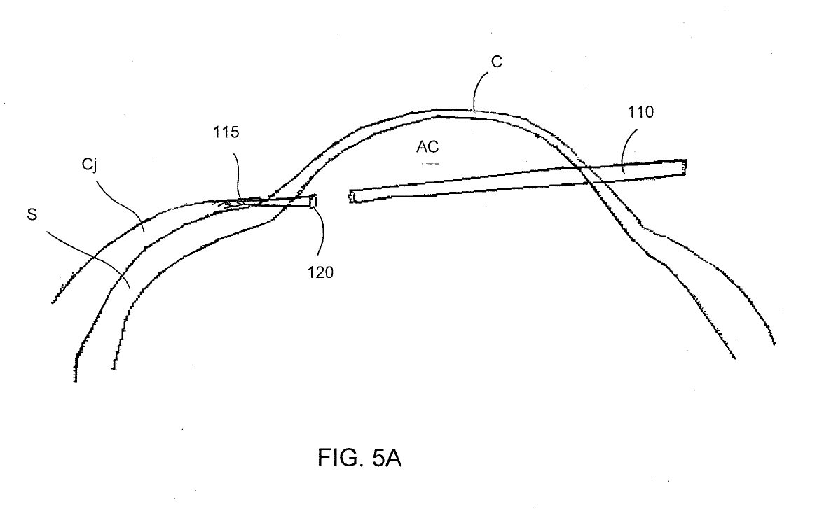

The branched internal lumen can serve as a passageway for the flow of aqueous humor through the implant 105 directly from one region of the eye into another region of the eye or another region outside the eye. For example, the implant 105 can shunt fluid from the anterior chamber to the suprachoroidal space. The, implant 105 can shunt fluid from the anterior chamber to a filtration flap or bleb. The implant can be implanted using a minimally-invasive procedure through the sclera S into the subconjunctival space. FIGS. 5A-5B show the branched implant 105 bridging between the anterior chamber AC to outside the sclera S. The distal ends 115 can be positioned just under the conjunctiva Cj. Implantation can be accomplished surgically such that the implant 105 tunnels through the sclera S but the conjunctiva Cj is not penetrated. In another embodiment, implantation can be performed from inside the subconjunctival space via the so-called ab interno approach. The internal lumen can also be used as a pathway for flowing irrigation fluid into the eye generally for flushing or to maintain pressure in the anterior chamber, or using the fluid to hydraulically create a dissection plane into or within the suprachoroidal space.

As mentioned above, the branched implant 105 can resist migration in both the proximal direction (arrow P) and distal direction (arrow D) within a tissue channel (see FIG. 2C). Removal of the delivery tube allows the branches of the implant 105 to spread apart therein increasing the overall diameter of the implant 105 within the tissue channel. This expansion of the branches prevents movement of the implant 105 back through the tissue channel in the proximal direction. Further, the ends of the branches in their spread apart configuration can dig into the surrounding tissue therein preventing migration in the distal direction.

Additionally, the material of the branches can be softer compared to the surrounding tissues through which the device is inserted and prevent distal migration of the implant 105. For example, urging the expanded, branched implant 105 in a distal direction can result in the flexible branches bowing and bending up against the surrounding tissues. This bowing and bending of the flexible branches against the surrounding tissues can result in the region of expanded diameter near the distal end region of the implant such that any further movement in the distal direction between tissue layers is prevented.

### Implant Delivery System

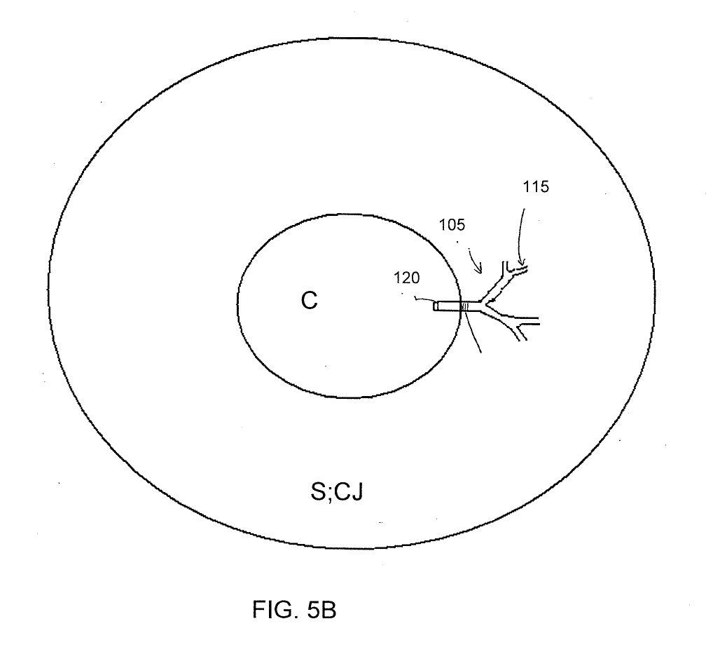

There are now described devices and methods for delivering and deploying the branched implant described herein into the eye. In an embodiment, a delivery system is used to deliver the implant into the eye. FIG. 6 shows an exemplary delivery system 905 that can be used to deliver the implant 105 into the eye. It should be appreciated that the delivery system 905 is exemplary and that variations in the structure, shape and actuation of the delivery system 905 are possible.

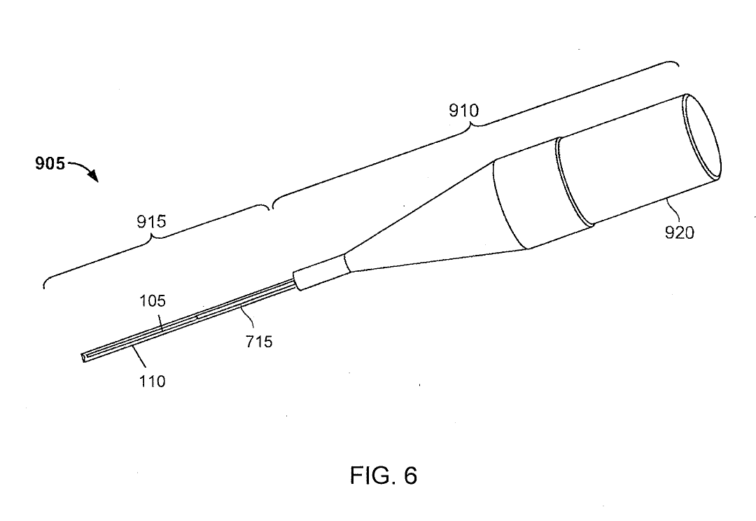

The delivery system 905 can include a handle component 910 that controls an implant placement mechanism, and a delivery component 915 that removably couples to the implant for delivery of the implant into the eye. The delivery component 915 can include an elongate sheath or delivery tube 110 (which was previously discussed above) that is sized and shaped to be inserted longitudinally over the implant 105. The elongate delivery tube 110 can be positioned over the implant during delivery to maintain or assist in maintaining the branches of the implant 105 in a low profile insertion configuration, as discussed above. The distal, branched end 115 of the implant can be positioned distally within the delivery tube 110 and the proximal, unbranched end 120 of the implant 105 can be positioned proximally within the delivery tube 110 (See FIG. 7A). In an embodiment, the branches of the implant at the distal end region can be gathered and held in place by an adjacent structure, such as a bullet nose (not shown) to provide a smooth insertion configuration that aids in the tunneling between tissue layers. It should be appreciated that a delivery wire can be used for delivery of the implant as well.

The delivery component 915 can also include a pusher 715 extending through a portion of the internal lumen of the delivery tube 110 that can abut the unbranched, proximal end region 120 of the implant 105 positioned within the delivery tube 110 (See FIG. 7A). Again with reference to FIG. 6, the handle component 910 of the delivery system 905 can be actuated to control delivery of the implant. In this regard, the handle component 910 includes an actuator 920 that can be actuated to cause relative sliding movement of the delivery tube 110 and the pusher 715. For example, the actuator 920 can be manipulated to cause the delivery tube 110 to withdraw proximally relative to the pusher 715 to release the implant 105 at the target location in the eye.

### Exemplary Methods of Delivery and Implantation

An exemplary method of delivering and implanting the implant into the suprachoroidal space using a minimally-invasive procedure is now described. It should be appreciated that the branched implant can be implanted such that fluid is also shunted from the anterior chamber to a surface bleb or filtration flap. The branched implant can also be implanted using a minimally-invasive procedure through the sclera S into the subconjunctival space. The branched implant 105 can bridge between the anterior chamber AC to outside the sclera S such that the branched, distal ends 115 are positioned just under the conjunctiva (as shown in FIGS. 5A-5B) and the unbranched, proximal end 120 is positioned in the anterior chamber AC. In another embodiment, implantation can be performed from inside the subconjunctival space via the so-called ab-interno approach.

In general, the implant can be implanted using a delivery system by entering the eye through a corneal incision and penetrating the iris root or a region of the ciliary body or the iris root part of the ciliary body near its tissue border with the scleral spur to create a low-profile, minimally-invasive blunt dissection in the tissue plane, for example between the sclera and the ciliary body or between the sclera and the choroid. The implant can be then positioned in the eye so that it provides fluid communication between the anterior chamber and the suprachoroidal space.

One or more implants 105 can be mounted on the delivery system 905 for delivery into the eye. The implant 105 can be mounted on the delivery system 905 such as by inserting the implant 105 through the lumen of a delivery tube 110. The unbranched, proximal portion 120 of the implant 105 can be positioned in a proximal region of the delivery tube 110 and the branched, distal end portion 115 of the implant 105 can be positioned in a distal-most region of the delivery tube 110. The eye can be viewed through a viewing lens or other viewing means in order to ascertain the location where the implant 105 is to be delivered. With reference to FIG. 7B, the delivery system 905 can be positioned such that the distal tip of the delivery tube 110 can penetrate through the cornea. In this regard, an incision can be made through the eye, such as within the limbus of the cornea. In an embodiment, the incision can be very close to the limbus, such as either at the level of the limbus or within 2 mm of the limbus in the clear cornea. The delivery tube 110 can be used to make the incision or a separate cutting device can be used. For example, a knife-tipped device or diamond knife can be used to initially enter the cornea. A second device with a spatula tip can then be advanced over the knife tip wherein the plane of the spatula is positioned to coincide with the dissection plane. Thus, the spatula-shaped tip can be inserted into the suprachoroidal space with minimal trauma to the eye tissue.

The corneal incision can have a size that is sufficient to permit passage of the implant therethrough. In this regard, the incision can be sized to permit passage of only the implant without any additional devices, or be sized to permit passage of the implant in addition to additional devices, such as the delivery device or an imaging device. In an embodiment, the incision can be about 1 mm in size. In another embodiment, the incision can be no greater than about 2.85 mm in size. In another embodiment, the incision can be no greater than about 2.85 mm and can be greater than about 1.5 mm. It has been observed that an incision of up to 2.85 mm is a self-sealing incision. For clarity of illustration, the drawing is not to scale.

The delivery tube 110 can approach the iris root IR from the same side of the anterior chamber AC as the deployment location such that the delivery tube 110 does not have to be advanced across the iris. Alternately, the delivery tube 110 can approach the insertion location from across the anterior chamber AC such that the delivery tube 110 is advanced across the iris and/or the anterior chamber toward the opposite iris root. The delivery tube 110 can approach the iris root IR along a variety of pathways. The delivery tube 110 does not necessarily cross over the eye and does not intersect the center axis of the eye. In other words, the corneal incision and the location where the implant is implanted at the iris root can be in the same quadrant (if the eye is viewed from the front and divided into four quadrants). Also, the pathway of the implant from the corneal incision to the iris root desirably does not pass through the centerline of the eye to avoid interfering with the pupil.

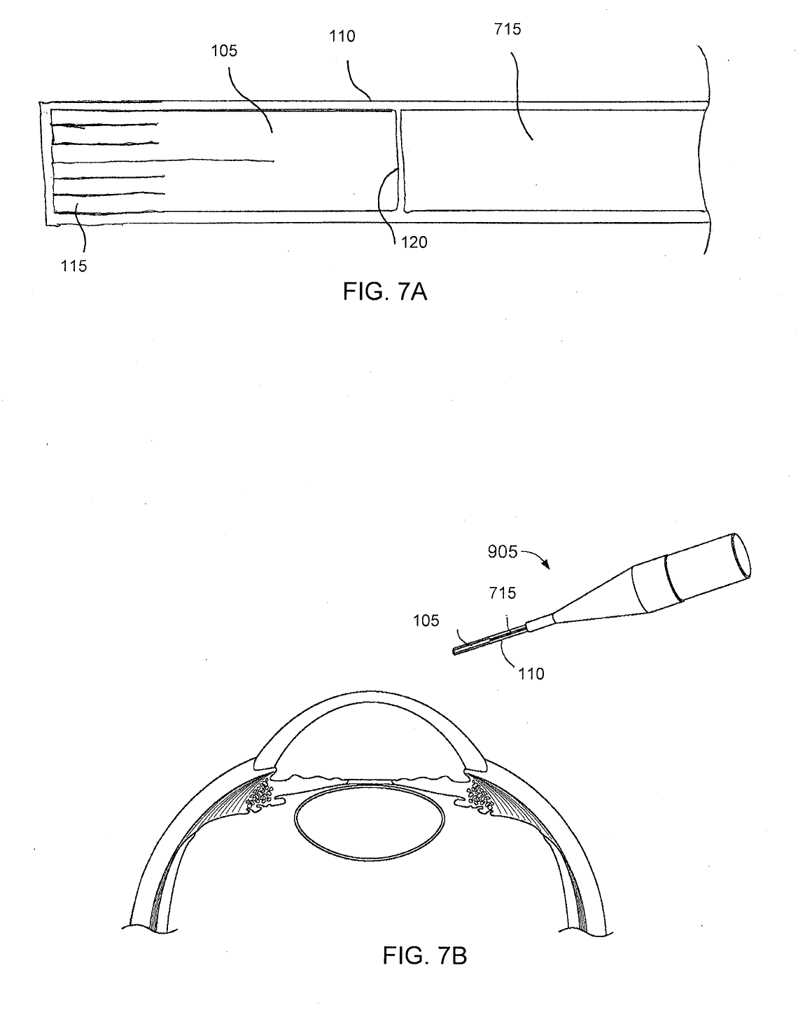

FIG. 8 shows an enlarged view of the anterior region of the eye. After insertion through the incision, the implant 105 mounted within the delivery tube 110 can be advanced through the cornea into the anterior chamber along a pathway that enables the implant to be delivered to a position such that the implant provides a flow passageway from the anterior chamber into the suprachoroidal space. The delivery tube 110 can travel along a pathway that is toward the scleral spur such that the delivery tube 110 passes near the scleral spur on the way to the suprachoroidal space. The scleral spur is an anatomic landmark on the wall of the angle of the eye. The scleral spur is above the level of the iris but below the level of the trabecular meshwork. In some eyes, the scleral spur can be masked by the lower band of the pigmented trabecular meshwork and be directly behind it. In an embodiment, the delivery tube 110 does not pass through the scleral spur during delivery. Rather, the delivery tube 110 abuts the scleral spur and then moves downward to dissect the tissue boundary between the sclera and the ciliary body, the dissection entry point starting just below (posterior) the scleral spur. The delivery tube 110 can penetrate the iris root or a region of the ciliary body or the iris root part of the ciliary body near its tissue border with the scleral spur. The combination of delivery tube 110 properties and the angle of approach allows the procedure to be performed “blind” as the instrument tip follows the inner curve of the scleral wall to dissect the tissue and create a mini cyclo-dialysis channel to connect the anterior chamber to the suprachoroidal space. The surgeon can rotate or reposition the handle of the delivery device in order to obtain a proper approach trajectory for the distal tip of the delivery tube 110, as described in further detail below. The delivery tube 110 can be pre-shaped, steerable, articulating, or shapeable in a manner that facilitates the delivery tube 110 approaching the suprachoroidal space along a proper angle or pathway.

As mentioned, the scleral spur is not necessarily penetrated during delivery. If penetration of the scleral spur is desired, penetration through the scleral spur can be accomplished in various manners. In one embodiment, a sharpened distal tip of the delivery tube 110 or the implant punctures, penetrates, dissects, pierces or otherwise passes through the scleral spur toward the suprachoroidal space. The crossing of the scleral spur or any other tissue can be aided such as by applying energy to the scleral spur or the tissue via the distal tip of the delivery tube 110. The means of applying energy can vary and can include mechanical energy, such as by creating a frictional force to generate heat at the scleral spur. Other types of energy can be used, such as RF laser, electrical, etc.

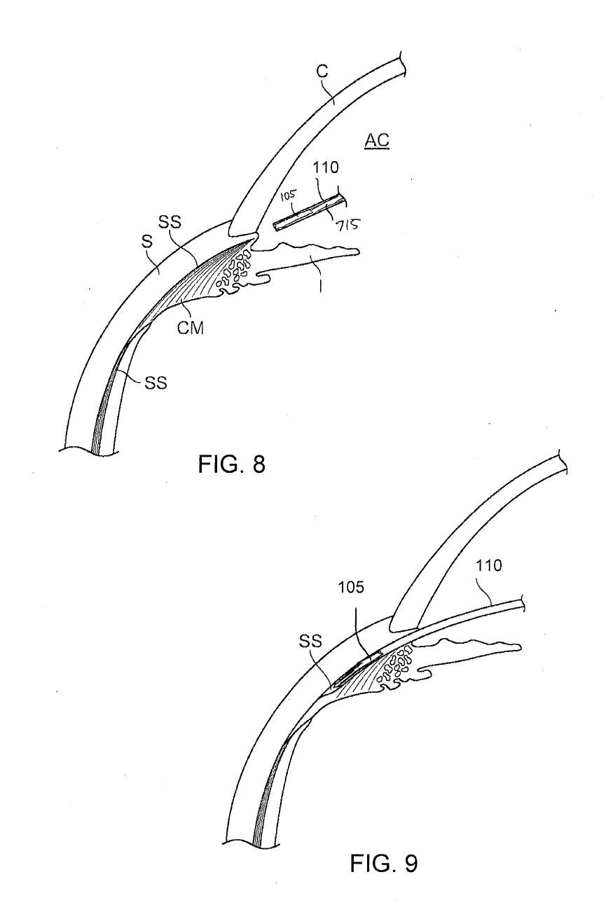

FIG. 9 shows the distal tip of the delivery tube 110 positioned within the suprachoroidal space SS. As the delivery tube 110 advances through tissue, the distal tip can cause the sclera to peel away or otherwise separate from the ciliary body or the choroid. As mentioned above, a variety of parameters including the shape, material, material properties, diameter, flexibility, compliance, pre-curvature and tip shape of the delivery tube 110 make it more inclined to follow an implantation pathway that mirrors the natural pathway between tissue layers, for example between tissue layers the sclera and choroid, and the curvature of the eye. The delivery tube 110 can be continuously advanced into the eye, until the distal tip is located at or near the suprachoroidal space such that the branched portion 115 of the implant 105 is positioned within the suprachoroidal space and the largest diameter (unbranched) portion 120 of the implant 105 is positioned within the anterior chamber. In one embodiment, at least 1 mm to 2 mm of the implant (along the length) remains in the anterior chamber. The implant 105 can then be released from the delivery tube 110 in the manner described above.

While this specification contains many specifics, these should not be construed as limitations on the scope of an invention that is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed.

What is claimed is:

1. A glaucoma treatment device comprising:
a tubular, unbranched portion having an internal flow pathway extending between a distal region and a proximal region of the unbranched portion, wherein the proximal region comprises at least one inflow port that is in fluid communication with the internal flow pathway; and
a branched portion extending from the distal region of the tubular, unbranched portion, wherein the branched portion comprises first and second reversibly deformable branches, wherein each of the first and second branches has at least one outflow port and an internal lumen in fluid communication with the flow pathway of the unbranched portion, and wherein each of the first and second branches are biased away from one another when in a relaxed state.

2. The device of claim 1, wherein the branched portion is defined by a fractal design.

3. The device of claim 2, wherein the fractal design comprises a self-repetitive fractal design.

4. The device of claim 1, wherein at least one of the first and second branches comprises a third and a fourth branch, wherein the third and fourth branches are reversible deformable, have at least one outflow port and an internal lumen in fluid communication with the flow pathway of the unbranched portion.

5. The device of claim 4, wherein the internal lumen of each of the third and fourth branches has a diameter that is smaller than a diameter of the branch from which it originated.

6. The device of claim 1, wherein the reversibly deformable branches can be constrained such that an outer dimension of the branched portion approaches an outer dimension of the unbranched portion.

7. The device of claim 1, wherein the inflow port is positioned in fluid communication with an anterior chamber of the eye and the at least one outflow port is positioned within a region outside the anterior chamber.

8. The device of claim 1, wherein the at least one outflow port is positioned at a distal end of the first and second branches.

9. The device of claim 1, wherein the at least one outflow port is positioned near a distal end of the first and second branches.

10. The device of claim 1, wherein at least one of the first and second branches has more than one outflow port.
